(19) United States
(12) Patent Application Publication
(10) Pub. No.: US 2024/0005766 A1
(43) Pub. Date: Jan. 4, 2024

(54) AUTOMATIC AND EFFICIENT FALL PREDICTION ASSESSMENT BASED ON MACHINE LEARNING AND A TRACKING SYSTEM

(71) Applicant: CARMEL HAIFA UNIVERSITY ECONOMIC CORPORATION LTD., Haifa (IL)

(72) Inventors: Hagit HEL-OR, Zichron-Yacov (IL); Ilan SHIMSHONI, Haifa (IL); Nadav EICHLER, Haifa (IL); Shmuel RAZ, Kfar Vradim (IL)

(21) Appl. No.: 18/216,297

(22) Filed: Jun. 29, 2023

Related U.S. Application Data
(63) Continuation of application No. PCT/IL2021/051561, filed on Dec. 30, 2021.
(60) Provisional application No. 63/132,576, filed on Dec. 31, 2020.
(60) Provisional application No. 63/257,929, filed on Oct. 20, 2021.

(51) Int. Cl. G08B 21/04; G06T 17/10

(52) U.S. Cl. CPC G08B 21/0446; G08B 21/0476; G06T 17/10

(57) ABSTRACT

A computer-implemented method for predicting risk of fall of a user is disclosed. The method includes: receiving temporal data related to a skeleton of the user, from at least two sensors; reconstructing from the temporal data at least one of, a temporal 3D scene reconstruction, and a temporal three-dimensional (3D) skeleton reconstruction; extracting spatio-temporal features from the 3D scene reconstruction or the 3D skeleton reconstruction; introducing at least one spatio-temporal feature to a machine-learning (ML) model, wherein said ML model is trained to predict fall probability of a user based on said spatio-temporal feature; and predicting fall probability of the user based on an output of the ML model.

### CROSS-REFERENCE TO RELATED APPLICATIONS

This application is a Bypass Continuation of PCT International Application No. PCT/IL2021/051561 having International filing date of Dec. 30, 2021, which claims the benefit of priority of U.S. Patent Application No. 63/132,576, filed Dec. 31, 2020, and U.S. Patent Application No. 63/257,929, filed Oct. 20, 2021, all of which are hereby incorporated by reference in their entirety.

### FIELD OF THE INVENTION

The invention generally relates to methods and systems for assessing the risk of fall. More particularly, the present invention relates to the field of automatic and digital efficient assessment of fall risk based on machine learning models.

### BACKGROUND

Accidental falls are the leading cause of injury-related death and hospitalization in old age with over one-third of adults over the age of 65 and almost 50% of older adults over the age of 80 experience at least one fall a year.

Falls are one of the main causes of disability, loss of independence, and reduced quality of life. It incurs high expenses on the individuals, their families, and the public health system. On the other hand, it has been shown that individuals can significantly reduce the risk of falls by participating in fall prevention programs. Considering that the elderly population is dramatically increasing in number, with expected elderly population (age 60 and older) reaching 22% worldwide by year 2050, with 35% in Europe, and 28% in North America, the necessity for fall risk assessment and intervention is critical.

Indeed, world-wide calls have been initiated for fall prevention, and fall risk assessment. To date, fall risk assessment is clinical, relying on the expertise of physiotherapists and medical experts for performing the diagnosis. The extent and severity of fall risk is quantified using standard scales. One of the most common, is the comprehensive f (BBS). BBS, shown in the images of FIG. 1, is a physical test, used to evaluate stability and fall risk. It includes 14 motor tasks that are evaluated by a medical professional. (Source: The Fall Prevention Center of Excellence (StopFalls.org)).

BBS includes 14 balance tasks, ranging from unsupported sitting and standing to reaching forward while standing and standing on one leg (FIG. 1). Performance of the task is graded on a 5-level scale ranging from zero (unable) to four (independent). The final BBS measure is the sum of all individual scores. The BBS has been validated and it has been shown that a score of 36 or less, indicates a near 100% chance of fall within 6 months. A a score of 0-20 is considered high fall risk, 21-40 medium fall risk, and 41-56 low fall risk.

Although comprehensive, it is a rigorous and time-consuming subjective examination. Currently, the BBS diagnosis relies on expensive and limited medical professional resources, strongly restricting the number of patients diagnosed and monitored. New and more efficient methods for fall prediction are necessary to initiate a community wide screening to identify older people at high risk of falling who would benefit from participating in fall prevention programs.

### SUMMARY OF THE INVENTION

Some aspects of the invention may be related to a computer-implemented method for predicting risk of fall of a user, comprising: receiving temporal data related to a skeleton of the user, from at least two sensors; reconstructing from the temporal data at least one of, a temporal 3D scene reconstruction, and a temporal three-dimensional (3D) skeleton reconstruction; extracting spatio-temporal features from the 3D scene reconstruction or the 3D skeleton reconstruction; introducing at least one spatio-temporal feature to a machine-learning (ML) model, wherein said ML model is trained to predict fall probability of a user based on said spatio-temporal feature; and predicting fall probability of the user based on an output of the ML model.

In some embodiments, training the ML model comprises: receiving a training dataset, comprising a plurality of spatio-temporal features, wherein the plurality of spatio-temporal features was received by: receiving a three-dimensional (3D) temporal data related to skeletons of a plurality of users, from the at least two sensors; reconstructing from the temporal data at least one of: a plurality of temporal 3D scene reconstructions, and a plurality of 3D skeleton reconstructions extracting the plurality spatio-temporal features from the plurality of 3D scene reconstructions or the plurality of 3D skeleton reconstructions; receiving a set of fall-related labels, corresponding to the plurality of spatio-temporal features; and training the ML model based on the training dataset, to predict fall probabilities, using the set of fall-related labels as supervisory data.

In some embodiments, each fall-related label includes a fall probability received from a professional via a user device. In some embodiments, each fall-related label is related to one or more spatio-temporal features received when a user was conducting at least one Berg Balance Scale (BBS) task. In some embodiments, the at least two sensors are selected from two video cameras located at different angles with respect to the user, one video camera and at least one positioning sensor located on a body of the user and an array of positioning sensors located on the body of the user.

In some embodiments, the 3D temporal data related to a skeleton of the user is received while the user is performing at least one BBS task. In some embodiments, the method may further include determining a number and type of additional BBS tasks the user is required to perform based on a fall probability given to the at least one BBS task. In some embodiments, the number and the type of the BBS tasks are determined such that an accuracy of the prediction is at least 85% with respect to a training dataset. In some embodiments, the method further comprises determining the order of performing the BBS tasks based on previously performed BBS tasks. In some embodiments, the at least one fall probability is predicated from 3D temporal data received while the user is performing up to 6 BBS tasks.

In some embodiments, the spatio-temporal features are selected from: relative position of skeleton joints, distance between body parts, angle at body joints, and height of joints from the ground.

Some aspects of the invention are directed to system for predicting risk of fall of a user, comprising: at least two sensors; and at least one computing device configured to: receive a temporal data related to a skeleton of the user, from the at least two sensors; reconstruct from the temporal data at least one of, a temporal 3D scene reconstruction and a temporal three-dimensional (3D) skeleton reconstruction; extract spatio-temporal features from the 3D scene reconstruction or the 3D skeleton reconstruction; introduce at least one spatio-temporal feature to a machine-learning (ML) model, wherein said ML model is trained to predict fall probability of a user based on said spatio-temporal feature; and predict at least one fall probability of the user based on an output of the ML model.

In some embodiments, the at least one computing device is configured to train the ML model to: receive a training dataset, comprising a plurality of spatio-temporal features, wherein the plurality of spatio-temporal features was received, by: receiving a three-dimensional (3D) temporal data related to skeletons of a plurality of users, from the at least two sensors; reconstructing from the temporal data at least one of, a plurality of temporal 3D scene reconstructions or a plurality of 3D skeleton reconstructions; extracting the plurality spatio-temporal features from the plurality of 3D scene reconstructions or the plurality of 3D skeleton reconstructions; receive a set of fall-related labels, corresponding to the plurality of spatio-temporal features; and train the ML model based on the training dataset, to predict fall probabilitys, using the set of fall-related labels as supervisory data.

In some embodiments, each fall-related label includes a fall probability received from a professional via a user device. In some embodiments, each fall-related label is related to one or more spatio-temporal features received when a user was conducting at least one Berg Balance Scale (BBS) task.

In some embodiments, the at least two sensors are selected from: two video cameras located at different angles with respect to the user, one video camera and at least one positioning sensor located on a body of the user and an array of positioning sensors located on the body of the user. In some embodiments, the 3D temporal data related to a skeleton of the user is received while the user is performing at least one BBS task. In some embodiments, the at least one computing device is configured to: determine a number and type of BBS tasks the user is required to perform based on a fall probability given to the at least one BBS task. In some embodiments, the number and the type of the BBS tasks are determined such that an accuracy of the prediction is at least 85% with respect to the training dataset. In some embodiments, the at least one computing device is configured to: determine the order of performing the BBS tasks based on previously preformed BBS tasks.

In some embodiments, the at least one fall probability is predicated from 3D temporal data received while the user is performing up to 6 BBS tasks. In some embodiments, the spatio-temporal features are selected from, relative position of skeleton joints, distance between body parts, angle at body joints, height of joints from the ground.

### BRIEF DESCRIPTION OF THE FIGURES

The patent or application file contains at least one drawing executed in color. Copies of this patent or patent application publication with color drawing(s) will be provided by the Office upon request and payment of the necessary fee.

Exemplary embodiments are illustrated in referenced figures. Dimensions of components and features shown in the figures are generally chosen for convenience and clarity of presentation and are not necessarily shown to scale. The figures are listed below.

FIG. 1 depicts images of BBS tasks;

FIG. 2A. shows a schematic block diagram of a system for predicting risk of fall of a user according to some embodiments of the invention;

FIG. 2B is a flowchart of a method of predicting risk of fall of a user according to some embodiments of the invention;

FIG. 2C is a flowchart of a method of training machine-learning (ML) to predict fall probability of a user according to some embodiments of the invention;

FIGS. 3A and 3B an image of a body pose in front of a camera (3A) and a 3D sensor array (3B) for measuring distances of points in a scene from which a skeleton representation of the body pose is produced and illustrate a validation of the haze-lines, according to some embodiments of the invention;

FIG. 4. is an illustration of a multi-camera tracking system according to some embodiments of the invention;

FIG. 5. is an illustration showing skeleton data used for computing spatio-temporal features in a recorded video frame according to some embodiments of the invention;

FIGS. 6A and 6B are illustrations of confusion matrixes comparing a predicted risk level and the true level according to some embodiments of the invention;

FIG. 7. is a schematic diagram of an ML model according to some embodiments of the invention;

FIG. 8. is a graph showing accuracy vs. an average number of BBS tests for different selector methods trained on the physiotherapist scoring according to some embodiments of the invention;

FIG. 9. is a graph showing accuracy vs. an average number of BBS tests for different selector methods trained on the automatic BBS scoring according to some embodiments of the invention;

FIG. 10. is a graph showing accuracy vs. average number of BBS tests for a different initial subset of tasks according to some embodiments of the invention;

FIG. 11. shows occurrence matrices depicting the ordering of BBS tasks in E-BB S. columns indicate the order in the E-BBS according to some embodiments of the invention;

FIG. 12. shows occurrence matrices depicting the ordering of BBS tasks in E-BBS trained on the automatic BBS scoring data according to some embodiments of the invention; and

FIG. 13 is a block diagram, depicting a computing device which may be included in a system for predicting the risk of fall of a user according to some embodiments of the invention.

### DETAILED DESCRIPTION

One skilled in the art will realize the invention may be embodied in other specific forms without departing from the spirit or essential characteristics thereof. The foregoing embodiments are therefore to be considered in all respects illustrative rather than limiting of the invention described herein. Scope of the invention is thus indicated by the appended claims, rather than by the foregoing description, and all changes that come within the meaning and range of equivalency of the claims are therefore intended to be embraced therein.

In the following detailed description, numerous specific details are set forth in order to provide a thorough understanding of the invention. However, it will be understood by those skilled in the art that the present invention may be practiced without these specific details. In other instances, well-known methods, procedures, and components have not been described in detail so as not to obscure the present invention. Some features or elements described with respect to one embodiment may be combined with features or elements described with respect to other embodiments. For the sake of clarity, discussion of same or similar features or elements may not be repeated.

Although embodiments of the invention are not limited in this regard, discussions utilizing terms such as, for example, “processing,” “computing,” “calculating,” “determining,” “establishing”, “analyzing”, “checking”, or the like, may refer to operation(s) and/or process(es) of a computer, a computing platform, a computing system, or other electronic computing device, that manipulates and/or transforms data represented as physical (e.g., electronic) quantities within the computer's registers and/or memories into other data similarly represented as physical quantities within the computer's registers and/or memories or other information non-transitory storage medium that may store instructions to perform operations and/or processes.

Although embodiments of the invention are not limited in this regard, the terms “plurality” and “a plurality” as used herein may include, for example, “multiple” or “two or more”. The terms “plurality” or “a plurality” may be used throughout the specification to describe two or more components, devices, elements, units, parameters, or the like. The term “set” when used herein may include one or more items.

Unless explicitly stated, the method embodiments described herein are not constrained to a particular order or sequence. Additionally, some of the described method embodiments or elements thereof can occur or be performed simultaneously, at the same point in time, or concurrently.

Disclosed herein are automated fall risk assessment and predicting system and method. In one embodiment, provided herein are system and method for screening of a user for fall risk. In one embodiment, provided herein are system and method for determining whether the user having risk of fall is suitable for participation in a fall prevention program.

In some embodiments, the system may include at least two sensors which may provide to a computing device temporal data related to a skeleton of the user. The two sensors may be included in a multi-depth-camera human motion tracking system. The multi-depth-camera human motion tracking system may include: two video cameras located at different angles with respect to the user, one video camera, and at least one positioning sensor located on the body of the user, and/or an array of positioning sensors located on the body of the user. In some embodiment, the multi-depth-camera human motion tracking system captures a user performing the Berg Balance Scale (BBS) tasks.

In one embodiment, provided herein are system and method which comprise trained machine learning module to predict fall probability of a user, by, for example, predicting patient BBS scores from data received from the two sensors.

In one embodiment, the system and method utilize data extracted from the cameras, and machine learning classifiers evaluate the performance of the tasks by the individual. In one embodiment, the system and method utilize machine learning tools to establish or develop fall risk predictors which enable reducing the number of BBS tasks required to assess fall risk to 2-12, 3-8, or 4-6 tasks without compromising the quality and accuracy of the BBS assessment. In one embodiment, the system and method utilize machine learning tools to establish or develop fall risk predictors which enable increasing the quality and/or accuracy of the BBS assessment.

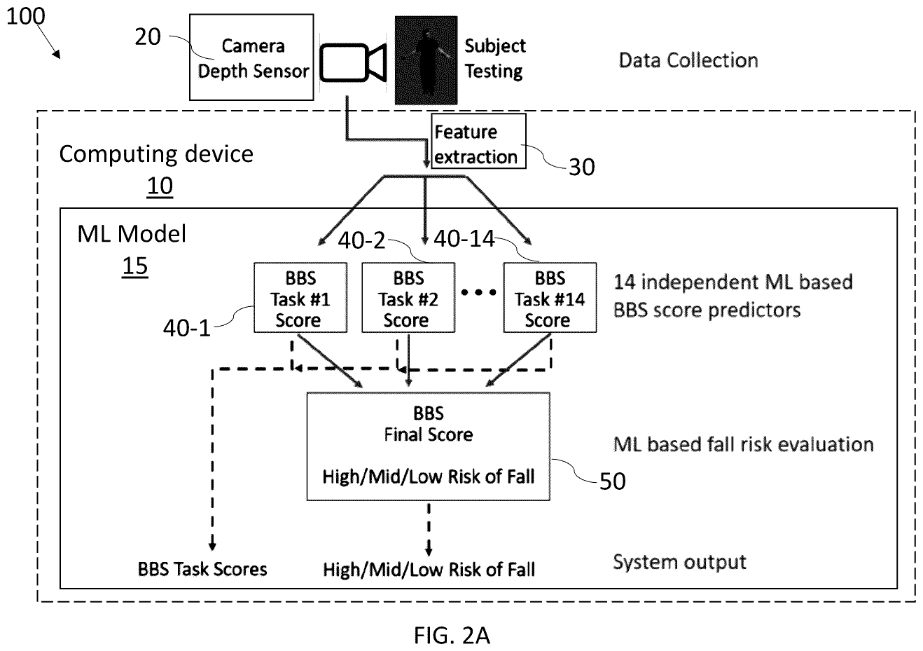

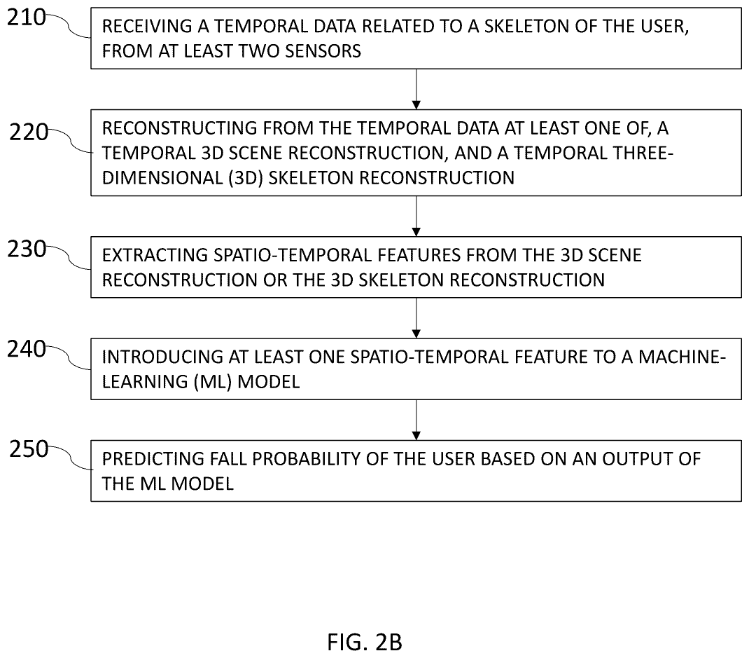

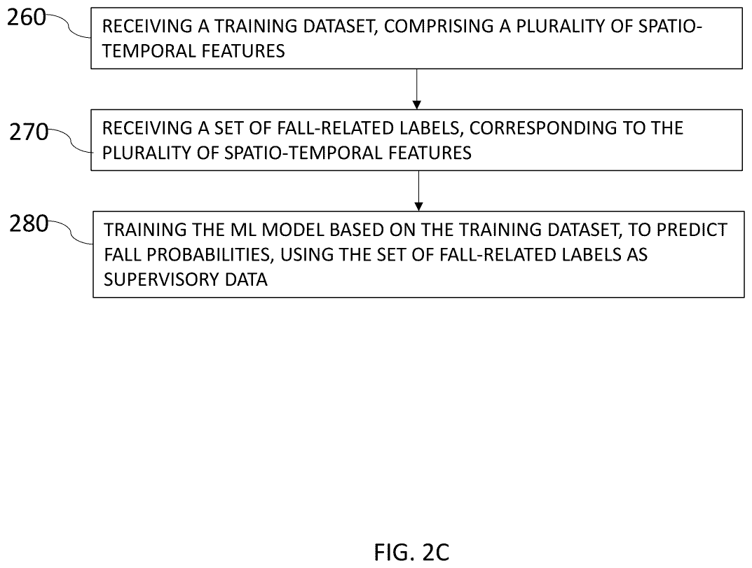

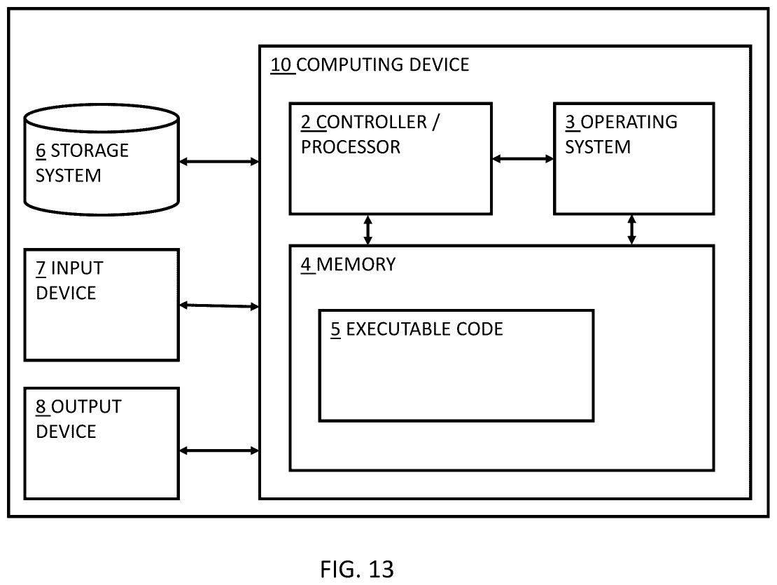

Reference is now made to FIG. 2A which shows a schematic block diagram of a system for predicting the risk of fall of a user according to some embodiments of the invention. System 100 may include computing device 10, which its hardware components are illustrated and discussed in detail with respect to FIG. 13, and at least two sensors 20. The software elements of system 100 are discussed with respect to the flowcharts of FIGS. 2B and 2C. The executable code (e.g., executable code 5 shown in FIG. 13) of computing device 10 may include a machine learning (ML) model 15.

In some embodiments, at least two sensors 20 may be selected from, two video cameras located at different angles with respect to the user, one video camera and at least one positioning sensor located on the body of the user and/or an array of positioning sensors located on the body of the user. In one embodiment, the system comprises an array of depth sensors. In one embodiment, the system comprises an array of ROS-compatible 3D sensors. In one embodiment, the system comprises an array of depth sensors for obtaining a 3D representation of a given skeleton of a user.

In some embodiments, the system comprises cameras capable of providing depth information of the scene. In one embodiment, the system comprises kinect 3D cameras (such as but not limited to: Kinect Azure). In one embodiment, the system comprises depth sensors that provide a distance from the camera for every point in the scene at each video frame. In one embodiment, the system comprises the time-of-flight component. In one embodiment, the data provided by the capture system is used to automatically construct a body pose representation in the form of a skeleton, where body joints are positioned in a 3D coordinate system relative to the camera.

Reference is now made to FIG. 2B which is a flowchart of a computer-implemented method for predicting risk of fall of a user according to some embodiments of the invention. The method of FIG. 2B may be performed by computing device 10 or by any suitable computer or processor.

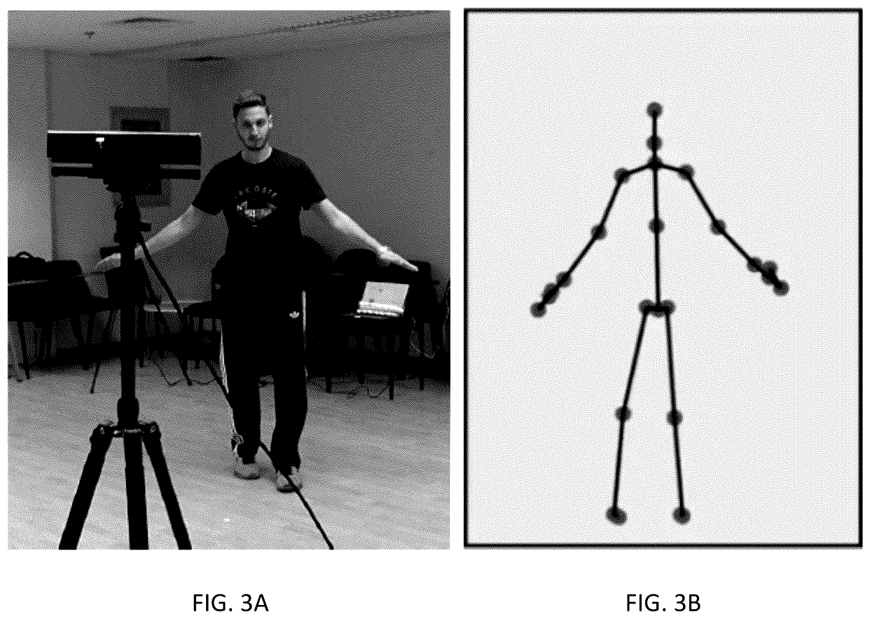

In step 210, temporal data related to a skeleton of the user is received from at least two sensors, for example, sensors 20. Two examples for such sensors are given in FIGS. 3A and 3B which include an image of a body pose in front of a camera (3A) and a 3D sensor array (3B). The data received from the camera or the sensor array may include distances of points in a scene from which a skeleton representation of the body pose is produced by illustrating and validating haze lines between the points, according to some embodiments of the invention.

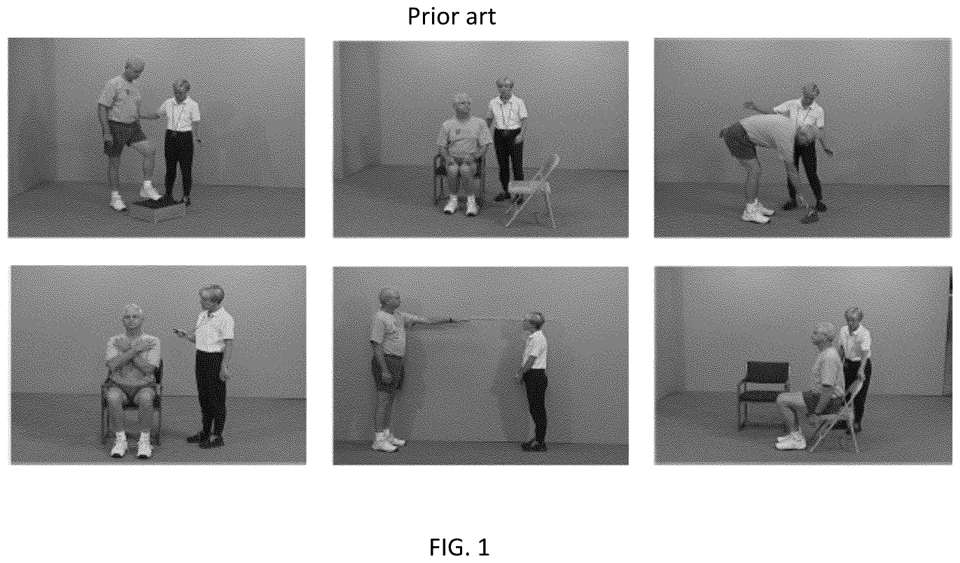

In some embodiments, the temporal data is captured when a user is performing at least one of the 14 BBS tasks, for example, at least one of the tasks shown in FIG. 1. In some embodiments, the temporal data is captured when a user other tasks, for example, tasks supervised by a professional.

In some embodiments, sensors 20 provide depth information of the scene. In one embodiment, sensors 20 provide a distance from at least one sensor to at least some of the points in the scene, for example, when sensors 20 are cameras, at each video frame. In one embodiment, the data provided by the capture system is used to automatically construct a body pose representation in the form of a skeleton, where body joints are positioned in a 3D coordinate system relative to the camera.

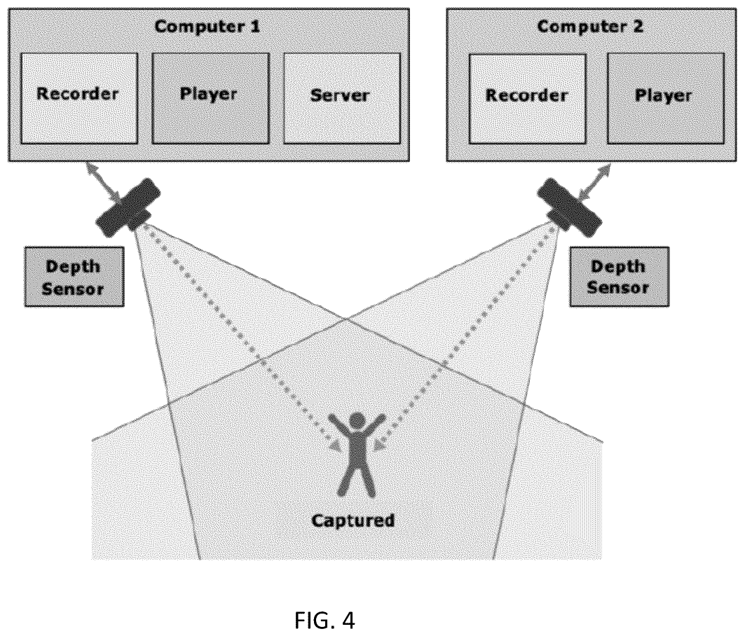

A nonlimiting example for capturing a full range of a patient's body motion using at least two cameras (e.g., sensors 20) is illustrated in FIG. 4. At least 2 depth-sensing cameras may be positioned two meters apart and directed approximately 45 degrees inward. In one embodiment, this setup allows integration of data from at least two cameras consequently reducing noise and skeleton errors.

A major drawback of any multi-camera system is the necessity of performing synchronization and calibration between the cameras. This process typically requires a designated calibration session with special calibration tools, a process that is often impractical and infeasible.

In one embodiment, the system comprising the at least two sensors 20 may perform synchronization and calibration automatically and on the fly by exploiting the patient's motion. In one embodiment, this system eliminates the need for calibration sessions, and create an easy to use system. In one embodiment, the automated calibration, allows integration of the skeletal data acquired by the at least two cameras as well as the additional data required for analysis such as the ground plane position and object location.

In one embodiment, the system tracks a user in the scene and outputs data per video frame: skeletal 3D joint positions, 3D point cloud data in the user's immediate surrounding, floor/ground position, and orientation, and a point cloud of objects in the scene relevant to the at least one BBS task.

In step 220, computing device 10 may reconstruct from the temporal data at least one of, a temporal 3D scene reconstruction, and a temporal 3D skeleton reconstruction. For example, computer device 10 may conduct at least some of the following data processing.

In one embodiment, a depth acquisition technique may be used. For example, computing device 10 may analyze depth data and/or may conduct fusion of depth data with an additional modality. In one embodiment, computing device 10 may provide 3D skeleton modeling. In one embodiment, computing device 10 may provide 3D object recognition. In one embodiment, computing device 10 may provide 3D biometrics. In one embodiment, computing device 10 may provide point cloud modeling and processing. In one embodiment, computing device 10 may provide individual/human action and/or motion recognition on depth data. In one embodiment, a storage system 6 of computing device 10 (illustrated in FIG. 13) may include depth datasets and benchmarks. In one embodiment, computing device 10 may provide depth data visualization. In one embodiment, computing device 10 may use computer vision technology. In one embodiment, computing device 10 may allow for constructing a 3D skeleton from 2D data.

In step 230, computing device 10 may extract spatio-temporal features (e.g., spatio-temporal features 30 in FIG. 2A) from the 3D scene reconstruction or the 3D skeleton reconstruction. In some embodiments, the spatio-temporal features are selected from: relative position of skeleton joints, the distance between body parts, the angle at body joints, the height of joints from the ground, and the like.

Reference is now made to FIG. 5 which is an illustration showing skeleton data used for computing spatio-temporal features in a recorded video frame according to some embodiments of the invention. In a nonlimiting example, when a video is used, for each video frame, spatio-temporal features may be extracted from the skeleton and 3D cloud point data. In some embodiments, all features are relative (to start position, to other body parts, to ground plane, etc.) and thus invariant to the position of the camera with respect to the subject/individual. In the nonlimiting example of FIG. 5, the head-hand distance and the shoulder angle were extracted.

Referring back to FIG. 2B, in step 240, computing device 10 may introduce at least one spatio-temporal feature 30 to ML model 15, wherein said ML model 15 is trained to predict fall probability of a user based on said spatio-temporal feature, as illustrated and discussed with respect to FIG. 2C. In a nonlimiting example, ML model 15 may recognize the BBS task performed by the user and may assign a score (40-1 to 40-14, illustrated in FIG. 2A) to the performed task. The assigned score may be use to calculate the fall probability of the user for each performed task.

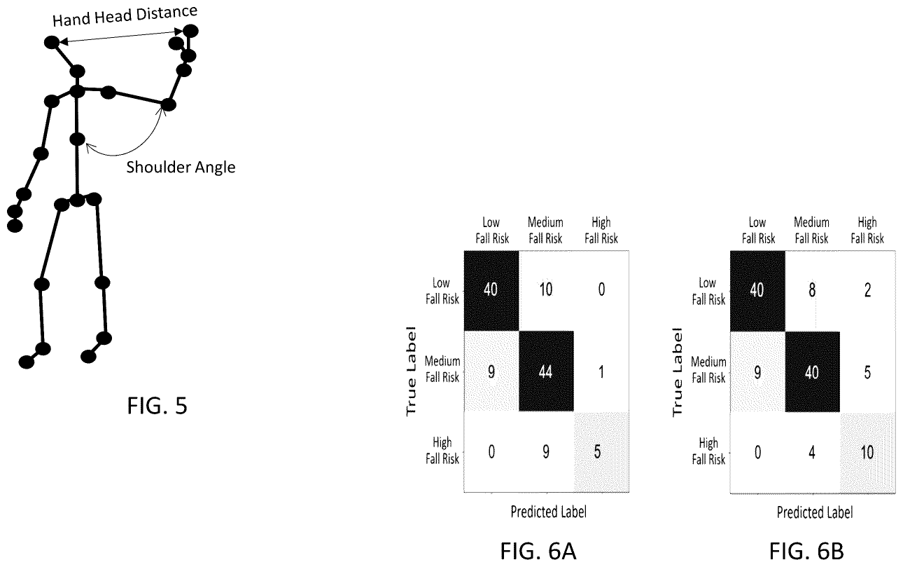

In step 250, computing device 10 may predict the fall probability of the user based on an output of ML model 15. For example, computing device 10 may calculate the final BBS score 50, illustrated in FIG. 2A, and based on this score determine if the user has a low, medium or high fall probability. Some nonlimiting examples for such calculation are given hereinbelow. A nonlimiting example for predicting the fall probability of the user based on BBS score is illustrated in FIGS. 6A and 6B.

FIGS. 6A and 6B are illustrations of confusion matrixes comparing a predicted risk level and the true level to some embodiments of the invention. The scores in FIG. 6A were determined using standard BBS thresholds for risk of fall and the scores in FIG. 6B were determined using thresholds that reduce false negatives. The mean square error (MSE) values were 0.25 for FIG. 6A and 0.29 for FIG. 6B which are both discussed in detail in the Examples section.

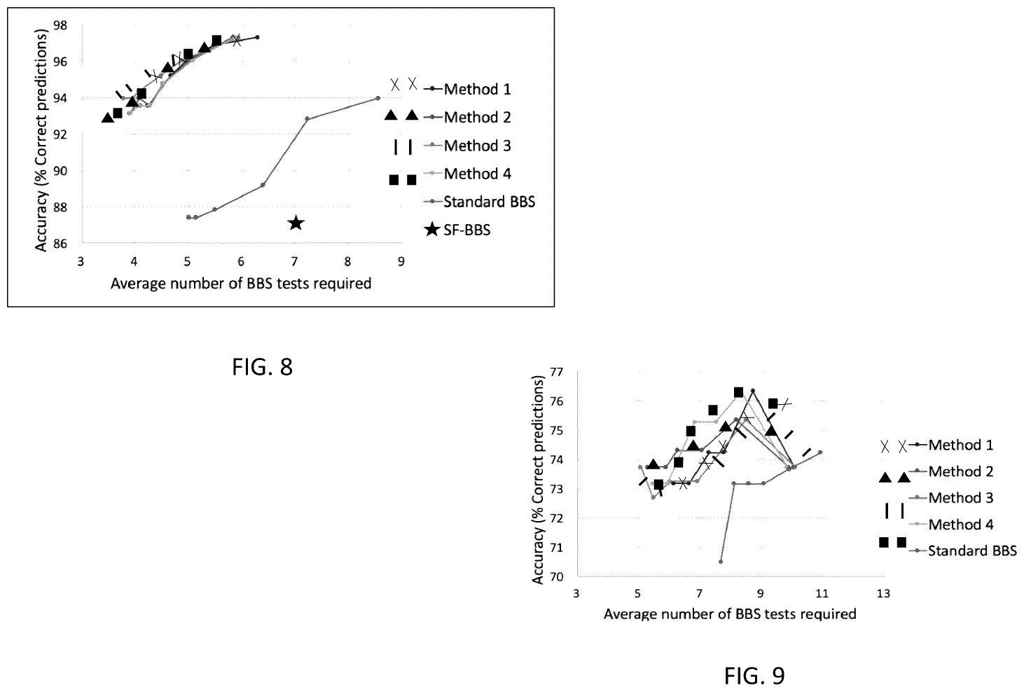

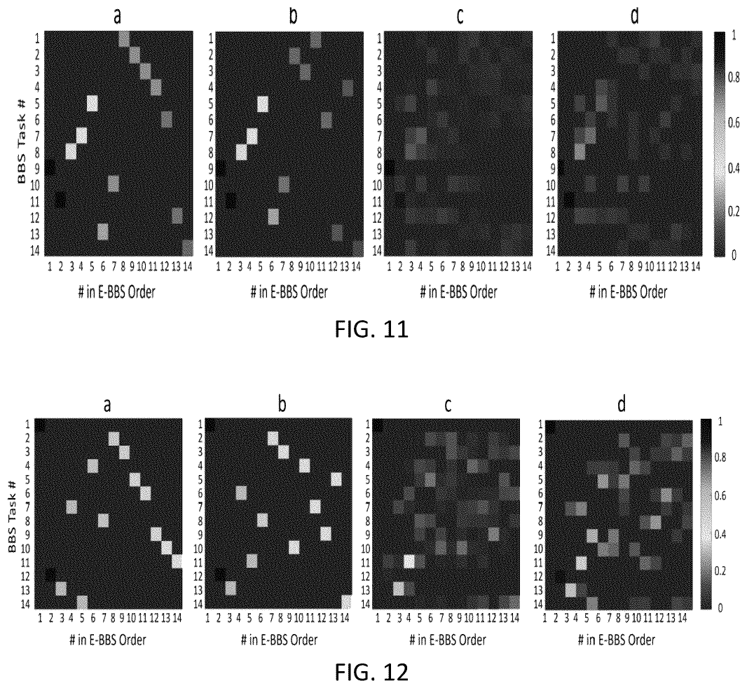

In some embodiments, the user may perform a first BBS task and computing device 10 may determine the number and the type of the BBS tasks are determined such that the accuracy of the prediction is at least 85% with respect to a training dataset (discussed with respect to FIG. 2C). Nonlimiting examples of determining the number of required BB S tasks are given in FIGS. 8, 9, and 10. In some embodiments, computing device 10 may further determine the order of performing the BBS tasks, as illustrated and discussed with respect to FIGS. 11 and 12 in the Examples section.

In some embodiments, the at least one fall probability is predicated from 3D temporal data received while the user is performing up to 6 BBS tasks. In one embodiment, machine learning is used to determine or evaluate fall risk predictors which enable reducing the number of BBS tasks required to assess fall risk, to 2-12 tasks. In one embodiment, machine learning is used to determine or evaluate fall risk predictors which enable reducing the number of BBS tasks required to assess fall risk, to 3-8 tasks. In one embodiment, machine learning is used to determine or evaluate fall risk predictors which enable reducing the number of BBS tasks required to assess fall risk, to 4-6 tasks. In one embodiment, machine learning is used to determine or evaluate fall risk predictors which enable reducing the number of BBS tasks required to assess fall risk, to 2-12 tasks. In one embodiment, machine learning is used to determine or evaluate fall risk predictors which enable reducing the number of BBS tasks required to assess fall risk without compromising the quality and accuracy of the BBS assessment.

Therefore, the reduced number of BBS tasks may lead to a more efficient BBS procedure termed herein as “Efficient-BBS (E-BBS)” which can be performed by physiotherapists in a traditional setting or deployed using system 100 and the method of FIG. 2B for more efficient and effective BBS evaluation.

In one embodiment, the E-BBS tasks are performed in either a predefined order or on a per-individual adaptive task program. In one embodiment, the use of E-BBS reduces the number of tasks per individual with a predictive accuracy of at least 90%, 92%, 95%, or 96%.

In one embodiment, EBBS is performed by system 100 allowing an efficient and effective BBS evaluation. In one embodiment, the automated system has a higher rate of fall risk predictions.

In one embodiment, the present invention includes a short form of the BBS (SFBBS). This test includes 7 of the 14 BBS tasks and the rating is on a 3-point scale (vs. the 5-point scale of BBS). The SFBBS was shown to have good validity, internal consistency, and reliability on stroke patients, on the elderly, and shown to compare well with the standard BBS.

In one embodiment, the present Efficient-BBS (E-BBS) significantly improves performance over the SFBBS. In one embodiment, the present invention utilizes a minimal number of BBS tasks that will maintain the accuracy of the standard BBS assessment while significantly reducing test time.

Reference is now made to FIG. 2C which is a flowchart of a method of training machine-learning (ML) model to predict the fall probability of a user according to some embodiments of the invention. The method of FIG. 2C may be performed by computing device 10 or by any other computing device.

In step 260, the computing device (e.g., computing device 10 or any other computing device) may receive a training dataset, comprising a plurality of spatio-temporal features, wherein the plurality of spatio-temporal features was received using steps 210-230 discussed herein above. Each spatio-temporal feature may be correlated with a specific performed BBS task.

In a nonlimiting example, for every user and for each of the 14 tasks, features were extracted to serve as ML training for, for example, for a Random Forest classifier. In one embodiment, for each video frame, spatio-temporal features were extracted from the skeleton and 3D cloud point data including relative position of skeleton joints, the distance between body parts, the angle at body joints, the height of joints from the ground and more (FIG. 5). All features were relative (to start position, to other body parts to ground plane, etc.) and thus invariant to the position of the camera with respect to the subject/individual.

In the nonlimiting example, features are measured in metric units. In one embodiment, from these per-frame features, global spatiotemporal features associated with the task's complete motion sequence are computed, including average speed, acceleration of joints, motion-paths, and maximal/minimal/mean values of the spatio-temporal features. In one embodiment, these global features served as the representation of each sample video for training the machine learning algorithm. In one embodiment, to optimize training of the system, feature selection was performed, by considering the most informative features as deduced by the trained ML model 15.

In one embodiment, the relevant features used in training and testing ranged between 50-400 or 100-200 dependent on the specific BBS task.

In step 270, the computing device may receive a set of fall-related labels, corresponding to the plurality of spatio-temporal features. For example, the level of the fall-related label may be given to the performed BBS task by a professional via a user device. In one embodiment, the BBS score assigned by the physiotherapist (e.g., a professional) to each subject in each video sequence (BBS task), served as the label associated with the sequence's feature vector.

In step 280, the computing device may train ML model 15 based on the training dataset, to predict fall probabilities, using the set of fall-related labels as supervisory data.

In a nonlimiting example, a separate Random Forest classifier was trained for each of the 14 tasks. The random forest consisted of 100 trees. The parameters for the classifiers were chosen using a grid search algorithm, which exhaustively searched through a manually specified subset of hyper-parameters. The predicted scores of the 14 classifiers 40-1 to 40-14 were then used to predict the final BBS fall risk level 50. Summing the physiotherapists' BBS scores per patient/individual, provided the correct labeling, with a score of 0-20 considered high fall risk, 21-40 medium fall risk, and 41-56 low fall risk. An SVM classifier was trained for the ternary fall-risk classification, whose parameters were also chosen using a grid search algorithm.

In the nonlimiting example, a radial basis function (RBF) was used as the SVM kernel, with a gamma coefficient of 1/nf, where of is the number of features, and a regularization parameter of value C=3. In one embodiment, the leave one-out-cross-validation method is utilized, leaving one subject/individual out on each iteration, to evaluate the accuracy of the classifiers. In one embodiment, this method also represents the classifiers' performance when given a single new subject to classify, rather than a batch of new subjects/individuals.

In some embodiments, ML model 15 may be used to reduce the number of BBS tasks required to be performed. In one embodiment, this approach reduces the number of tasks from 14 to an average of 4-6 tasks per subject, thus reducing the amount of time spent by the patient and the medical staff member (physiotherapist or the person supervising the automatic process) required for assessing fall risk. In one embodiment, this approach is applied both to the physical BBS and to the automatic system and in essence is exploited for any other battery of tests.

In one embodiment, 14 BBS tasks are scored and then summed or run through the algorithm in order to calculate the final fall risk assessment of the subject into one of the three classes (High, Medium, or Low fall risk). Considering the BBS assessment as an iterative process, every iteration can be considered as a “partial predictor” of the final fall risk assessment category. As more tests are performed and additional task scores are accumulated, the prediction becomes more accurate. Thus, ML is used to develop a method in which the BBS tasks are ordered in a manner that optimizes for accuracy of the final fall risk prediction and allows for the testing to terminate early when the prediction is at a high confidence level. The order of BBS tasks may be predetermined and constant across all subjects or may be adaptively determined per subject. Either way, the time for performing the BBS assessment is significantly reduced.

In some embodiments, a pre-processing step of building a dataset of fall risk predictors is conducted by computing device 10 or by any other computing device.

In one embodiment, the goal of the adaptive fall risk evaluation algorithm is to find the minimal subset of BBS tasks that will ensure the highest classification (prediction) accuracy for the risk of fall. In one embodiment, a dataset of fall risk predictors is used.

Consider all subsets of the 14 BBS tasks (2¹⁴−1 subsets), and for each subset, train a machine learning classifier to predict the final fall risk assessment using as input only the scores associated with the tasks in the subset. Together with the prediction, each classifier also outputs a measure of confidence in the prediction.

The fall risk predictors were trained using the patient data collected for the automated BBS system as further described. Two different datasets of predictors recreated. One dataset consisted of predictors trained on the physiotherapists' BBS scores with the ground truth risk category determined by the sum of these scores. The second dataset consisted of predictors trained on the BBS scores computed by the current automated BBS assessment system as described.

The fall risk category determined by the physiotherapists served as the ground truth in this case as well. Three types of machine learning algorithms were tested: SVM, Decision Trees and Random Forest. Each of these algorithms outputs the predicted risk class as well as the confidence in the prediction. The predictors in each dataset were ranked according to the accuracy in prediction (proportion of correct fall risk predictions) as well as the average confidence of the predictions over the training set. According to the empirical tests performed, the Random Forest model produced the most accurate predictors both in terms of accuracy and in terms of the average confidence level. Random Forests were trained with 100 trees that were built using three folds, with batch size 100, and confidence factor 0.25.

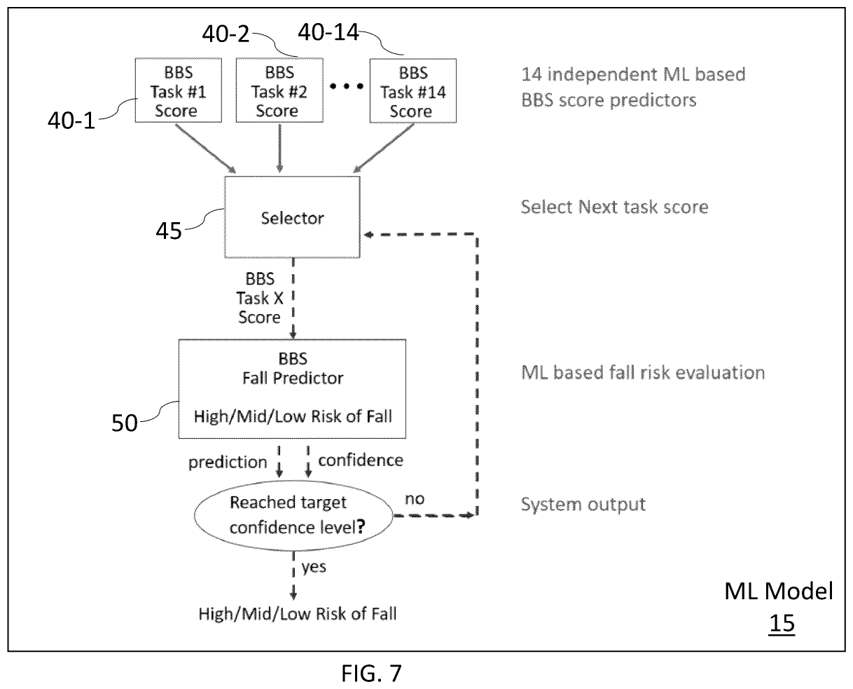

In some embodiments, computing device 10 may efficiently reorder the BBS task. The enhancement to BBS testing may involve re-ordering of the BBS tasks and interactively predicting the risk of falls after each task. Together with the fall risk prediction, the confidence in the prediction is outputted after each task as well. Given a confidence threshold, the BBS testing terminates when the prediction confidence exceeds the threshold. A schematic diagram of the system is shown in FIG. 7.

The new ordering and shortened sequence of BBS tasks is termed Efficient-BBS (E-BBS). The algorithm for determining the BBS task order may require: a) the first BBS task (or a subset of initial tasks) and b) a method to determine the next BBS task to perform. Denote by xi—the BBS scores of the i^{th} subject in the training set and by yi the risk class of xi (assume there are N such pairs (xi,yi)). Let CSS be the current sub-set of BBS tasks (tasks that have been performed) and denote by NT the next task to be determined from among the unused set of tasks UT. The pre-processing step may create a dataset of predictions for each sub-set of BBS tasks based on a training set of patient BBS scores and their risk category (as determined by the physiotherapists or by the automatic BBS system).

These predictors may be associated with a ranking according to their prediction accuracy. Thus the function Pred(SS; x) returns the risk class prediction for xi according to the trained predictor associated with the task subset SS. The function Conf(SS; x) returns the confidence associated with the prediction.

Four different selector methods, illustrated in FIG. 7, were developed and tested for choosing the next BBS task to be performed:
1) The next task NT is selected, based on equation 1, as the one augmented to CSS creates a subset whose predictor has the highest accuracy over the complete training set.

$$NT = arg {max}_{T ∈ UT} {\sum }_{i = 1}^{N} 𝕀 ( Pred ( \{ CSS , T \} , {x}_{i} ) = {y}_{i} ) , \qquad ( 1 )$$

where II is the indicator function.
2) NT may be determined using the accuracy score of the augmented subset predictor calculated only on the training examples xi for which the CSS predictor gives a confidence below the confidence threshold ST, using equation 2.

$$( 2 )$$

$$NT = arg {max}_{T ∈ UT} \text{?} 𝕀 ( Pred ( \{ CSS , T \} , {x}_{i} ) = {y}_{i} ) \times 𝕀 ( Conf ( CSS , {x}_{i} ) < ST ) ,$$

$$\text{?} \text{indicates text missing or illegible when filed}$$

3) The third applicable method is an adaptive method that depends on the patient being tested for BBS. NT may be determined as above but the i^{th} training example's contribution to the sum is weighted by its similarity to the scores q of the patient. The greater the similarity, the higher the weight, using equation 3.

$$NT = arg {max}_{T ∈ UT} {\sum }_{i = 1}^{N} 𝕀 ( Pred ( \{ CSS , T \} , {x}_{i} ) = {y}_{i} ) \times 𝕀 ( Conf ( CSS , {x}_{i} ) < ST ) \times d ( CSS ( {x}_{i} ) , CSS ( q ) ) . \qquad ( 3 )$$

where CSS(xi) and CSS(q) are the BBS scores of the patient and of the i^{th} training data on the tasks in CSS. As a similarity measure, we use d(l,k)=eXp(−∥l−k∥²/σ²) where the parameter controls the contribution of the point as a function of the distance.
4) The fourth methods may extend the third method by considering only the examples in the training set for which the algorithm correctly classified the example, using equation 4.

$$NT = arg {max}_{T ∈ UT} {\sum }_{i = 1}^{N} 𝕀 ( Pred ( \{ CSS , T \} , {x}_{i} ) = {y}_{i} ) \times 𝕀 ( Conf ( CSS , {x}_{i} ) < ST ) \times d ( CSS ( {x}_{i} ) , CSS ( q ) ) \times 𝕀 ( {y}_{i} = {\hat{y}}_{i} ) . \qquad ( 4 )$$

where \hat{ }yi is the final prediction of the algorithm i.e. Pred(AT; xi)=\hat{ }yi, where AT is the set of all tasks.

It can be seen that the first two selector methods produce a task sequence that is independent of the patient input. Thus, these selector methods produce a constant order of BBS tasks that is later used on all patient data when testing. Selector methods 3 and 4 are adaptive, as the NT task is chosen based on specific training data that is dependent on patient data being tested.

Thus for each patient data, a different BBS sequence of tasks is produced. In fact, all E-BBS sequences have the same initial part of the sequence.

In one embodiment, a patient, a subject, or an individual are used synonymously. In one embodiment, a patient, a subject, or an individual are used synonymously and is/are a human patient, a human subject, or a human individual. In one embodiment, a system and a method as described herein includes a multi depth-camera human motion tracking system integrated with machine learning (ML) algorithms. In one embodiment, ML is used to determine Efficient-BBS (E-BBS) battery of tests requiring the patient to perform a reduced subset of the BBS tests, while achieving the same or better quality of prediction as the full BBS test in a significantly shorter time.

In one embodiment, the invention includes any order of BBS tests. In one embodiment, the order of BBS tests is from easy challenge to advanced challenge. In one embodiment, the order of BBS tests is mixed thus easy challenges and advanced challenges.

In one embodiment, additional ML obtained data improves the performance of the adaptive variants of the E-BBS.

### EXAMPLES

### Example 1: Automatic BBS Score Prediction—Results

The classification performance of the automated BBS score prediction into the five score classes (0-4) was evaluated for each task. Additionally, the final risk assessment into the three classes of high, medium and low risk of fall was evaluated.

TABLE I
Automatic prediction of BBS scores per task

| | | | Samples per | Chance | | |
|---|---|---|---|---|---|---|
| Task | Task Description | N | Class <0, 1, 2, 3, 4> | Level | Accuracy | MSE |
| 1 | Sitting to Standing | 102 | 0, 0, 0, 66, 36 | 65% | 82% | 0.18 |
| 2 | Standing Unsupported | 111 | 0, 0, 15, 24, 72 | 66% | 72% | 0.36 |
| 3 | Sitting with Back Unsupported | 112 | 0, 0, 0, 0, 0, 112 | 100% | 100% | 0.0 |
| 4 | Standing to Sitting | 105 | 0, 0, 0, 53, 52 | 50% | 85% | 0.15 |
| 5 | Transfers | 96 | 0, 0, 22, 39, 35 | 41% | 73% | 0.36 |
| 6 | Standing Unsupported Eyes Closed | 101 | 0, 0, 0, 49, 52 | 51% | 68% | 0.32 |
| 7 | Stading Unsupported Feet Together | 106 | 13, 13, 0, 33, 47 | 44% | 72% | 0.37 |
| 8 | Reaching Forward | 75 | 0, 17, 0, 24, 34 | 45% | 69% | 0.51 |
| 9 | Pickup Object from the Floor | 99 | 7, 0, 0, 39, 53 | 54% | 72% | 0.31 |
| 10 | Look Behind Shoulders | 102 | 7, 9, 8, 32, 46 | 45% | 52% | 1.25 |
| 11 | Turn 360° | 100 | 14, 26, 20, 7, 33 | 33% | 66% | 0.60 |
| 12 | Alternate Feet on Step | 93 | 39, 11, 12, 0, 31 | 42% | 75% | 0.34 |
| 13 | Standing Unsupported One Foot in Front | 93 | 30, 14, 30, 0, 19 | 32% | 74% | 0.54 |
| 14 | Standing on One Leg | 109 | 39, 40, 8, 0, 22 | 37% | 66% | 0.80 |

Table I shows the accuracy in predicting the score of each BBS task. N is the number of samples tested in each task (differences between tasks are due to patients not completing some of the BBS tasks, or technical failures in some of the recordings) and the number of samples for each of the five classes. The table also shows the chance level since the distribution of samples was not evenly spread across the classes (some BBS tasks are very easy and are never scored low, e.g. Task #3 Sitting with Back Unsupported). The Table also shows the Mean Square Error (MSE) for misclassifications. The low MSE values indicate that when erred, the classification error was at most one score unit.

Results show that accuracy is always above chance, though low performance is achieved for some of the more challenging tasks, mainly due to the intervention of the physiotherapists to protect the patient from falling. These accuracies are, however, balanced out in the overall 3-way fall risk assessment.

Referring back to FIG. 6A which shows the confusion matrix between the predicted risk level and the true level determined by the sum of BBS task scores assessed by the physiotherapist. The accuracy in determining the overall 3-way level of fall risk. The success rate is at 75.5%, with a Mean Square Error (MSE) of 0.25. However, when assessing risk of falls, false negatives (FN) should be minimized.

Referring back to FIG. 6A which shows the resulting confusion matrix when this approach was adopted. The matrix shows that 9 high-risk samples were categorized as medium risk. The level of FN was controlled by optimizing ML model 15 for different thresholds while still maintaining a good level of success.

It can be seen that FN was reduced to 4 samples, however, at the expense of an increased number of false positives and a small increase in MSE to 0.29. The intent is to allow the physicians to select the level of accuracy, and achieve a satisfying false negative percentage as well as a satisfying overall accuracy.

Feature ranking was performed to determine the tasks most influencing the final risk of fall within the fall risk classifier.

In a nonlimiting example, features were scored by their F-statistic using Analysis of Variance (ANOVA) F-test algorithm [75], The following five BBS tasks were found to contributed the most to the classifier output (in decreasing order):
Turn 360_(Task #11)
Alternate Feet on Step (Task #12)
Transfers (Task #5)
Reaching forward with outstretched arm (Task #8)
Standing with Feet Together (Task #7)

The physiotherapists indeed confirmed that they consider the first two as the major contributors to the BBS evaluation.

### Statistical Analysis

Statistical analysis was performed to evaluate the correlations between the physiotherapist scores of the BBS and the predicted scores produced by system 100 (using ML model 15). Two physiotherapists scored each of the patients performing the 14 BBS tasks. For each patient, an ML prediction was calculated for each BBS task. The overall level of risk is categorized into 3 risk levels: high, medium and low risk of fall. The overall level of risk of fall is determined by the sum of the 14 scores: 0-20: high fall risk, 21-40: medium fall risk and 41-56 low fall risk.

An intraclass correlation (2-way mixed-model, single measure) was used for measuring interrater reliability of the BBS final score between the two physiotherapists and the machine learning (ML) prediction. Also included in the analysis is the minimal score between the two physiotherapists (MIN(A,D)), calculated on each sample independently.

This is in accord with a conservative scoring that tends toward fewer false alarms (see Section V-B). Intraclass correlation coefficient (ICC) above 0.8 reflects high reliability, 0.6-0.79 moderate reliability and less than 0.6, low reliability.

TABLE II
includes the intra- class correlation coefficient
between physicians and ML of BBS scores.

| | D | Min(A, D) | ML Prediction |
|---|---|---|---|
| A | .981* | .989* | .839* |
| D | | .992* | .834* |
| Min(A, D) | | | .824* |

Table II shows the ICC results. The ICC measure of raters' consistency in measuring final BBS scores is higher between the physiotherapists than between the physiotherapists and the ML prediction (Table II). The correlation between prediction results and physiotherapists' measures is high (>0:83) both between the two physiotherapists and between each physiotherapist and the ML prediction.

### Example 2: Efficient BBS

Given a starting subset of BBS tasks, a confidence threshold and a training set, each of the four selector methods produces a different optimal ordering of BBS tasks.

To evaluate the performance of each such ordering, a 5-fold cross-validation was used on the training set. For consistency, the results were compared with the standard ordering of BBS tasks as well as the Short Form BBS (SFBBS).

The quality of the performance of a specific ordering of tasks is evaluated using 2 parameters: the accuracy of predicting the fall risk category and the average number of BBS tasks required to complete the prediction process. Since the Efficient-BBS assessment terminates the testing when the confidence of the prediction reaches the desired threshold, the number of required BBS tasks is significantly lower than the number of BBS tasks in the standard BBS test.

The performance of the adaptive ordering was compared across selector methods, using confidence thresholds of 90, 92, 94, 96, 98, and 100. The initial subset of BBS tasks considered are of size 1, 2 and 3. Finally, the results were compared across the two types of datasets: based on the physiotherapist scoring and based on the automatic BBS scoring.

Referring now to FIG. 8. Which is a graph showing accuracy vs. an average number of BBS tests for different selector methods trained on the physiotherapist scoring according to some embodiments of the invention. The graph plots the accuracy and the average number of BBS tasks required for the E-BBS ordering produced by the four selector methods trained on the physiotherapists scoring as well as the standard BBS ordering. Plots show the performance across the different confidence thresholds.

The initial test set was the optimal set as will be discussed below and includes the 3 BBS tasks numbered 8, 9, 11g. As can be seen, all orderings of BBS tasks reach an accuracy of around 97% correct risk of fall predictions.

However, the different selector methods show a significant reduction in average BBS tasks compared to the standard BBS, requiring between 4-6 tasks on average compared to the 14 tasks of the standard BBS.

Additionally, the performance of the SF-BBS with 7 BBS tasks was plotted at an accuracy rate of 87% on patient data, showing that the E-BBS significantly outperforms the SF-BBS1. The four selector methods show comparable performance with a slight advantage for method 3.

Referring now to FIGS. 8 and 9. FIG. 8. is a graph showing accuracy vs. an average number of BBS tests for different selector methods trained on the physiotherapist scoring according to some embodiments of the invention, and FIG. 9. is a graph showing accuracy vs. an average number of BBS tests for different selector methods trained on the automatic BBS scoring according to some embodiments of the invention.

Both FIG. 8 and FIG. 9 display the same results when training is performed on the scores predicted by a physiotherapist scoring and an automatic BBS system, respectively. One can observe a lower rate of performance but, as before, the standard BBS is strongly outperformed by the four selector methods with method 4 showing the best performance. However, in this case, all methods reach an accuracy of 76%-77% correct risk of fall classification. Furthermore, it can be observed that there is a drop in accuracy when the confidence threshold reaches 100. This is due to the fact that the automatic BBS score assessment is inconsistent in its performance with some of the BBS tasks showing low prediction accuracy as shown in Table I.

The trained predictors select the high accuracy tasks first in the E-BBS ordering, leaving those with low accuracy to later in the ordering. When the confidence threshold is low, the BBS assessment of a subject is able to predict confidently without relying on those BBS tasks with low accuracy.

However, when confidence threshold reaches 100, those tasks must be recruited, and their inaccuracy leads to incorrect predictions of the overall fall risk. Albeit, this fault, the average number of required BBS tasks is still significantly lower than 14. We note that when continuing all the way to the 14th task, the four selector methods do not improve in accuracy beyond that shown in the plot, which is consistent with the non-adaptive results shown in FIG. 6.

The initial BBS tasks used by the E-BBS test was questioned. The reason for allowing a definition of an initial set of BBS tasks, is twofold. First, the iterative method of BBS testing and the design of the selector methods inherently imply that the optimal ordering was determined following a greedy algorithm.

As such, a local minimum may be reached in the optimization. To mitigate this effect, we allow a global optimal subset to be chosen as the first tasks in the ordering. Another advantage to the initial subset of tasks, is the flexibility in incorporating constraints and requirements in the ordering as may be defined by the medical professionals. For example, requiring the initial tasks in the testing to be easy tasks, and leaving the challenging tasks to later in the task order.

Without any external constraints on the initial task set, the set to be that which performs optimally was chosen. Since, the predictors trained in the pre-processing stage are each ranked by their prediction accuracy, the subset of predefined size whose predictor shows the best accuracy was chosen. Subsets of size 1, 2 and 3 were considered.

TABLE III
BBS tasks single predictors accuracy

| BBS Task | Accuracy |
|---|---|
| 9 | 85.5 |
| 7 | 81.4 |
| 6 | 81.2 |
| 11 | 80.8 |
| 8 | 80.0 |
| 4 | 77.8 |
| 5 | 77.4 |
| 12 | 76.2 |
| 1 | 74.2 |
| 10 | 72.6 |
| 2 | 70.7 |
| 13 | 67.5 |
| 14 | 67.3 |
| 3 | 50.8 |

Table III, shows the accuracy of the predictors associated with subsets of size 1 when trained on the physiotherapists scoring. The results in the table can be interpreted as the predictive quality of each individual task of the BBS. It can be seen that task 9, as a single task, is the best predictor of fall risk on our test set.

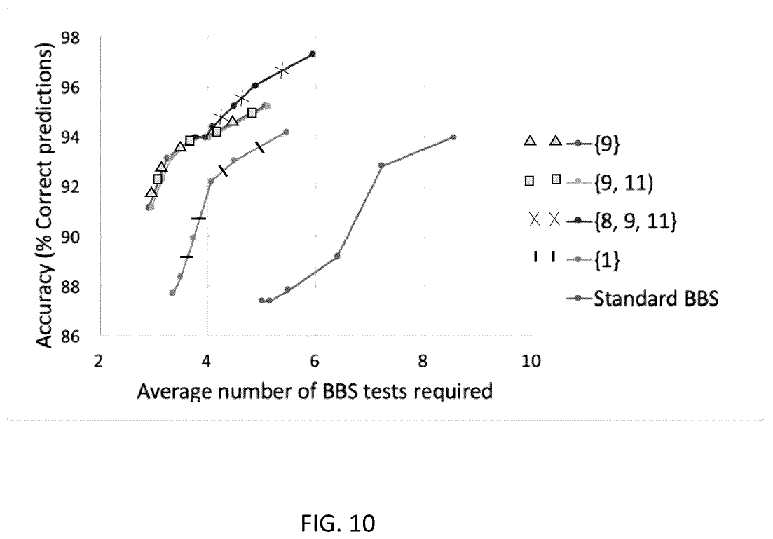

Similarly, for subsets of size 2 and 3, we find that the optimal initial task sets are f9,11g and f8,9,11g, as shown in FIG. 10. FIG. 10. is a graph showing accuracy vs. average number of BBS tests for a different initial subset of tasks according to some embodiments of the invention.

For comparison, also shown are the results for subset f1g and for the standard BBS test. Results are shown for the selector method 3. As can be seen, all E-BBS orderings are significantly better than the standard BBS and also better than the f1g subset case.

The accuracy is highest for the subset of size 3, reaching 97% accuracy at confidence level 100. All orderings require only 3-6 BBS tasks on average. Using BBS task 1 as the initial task, as is used in the standard BBS test, shows the least accurate results of the E-BBS orderings.

This is indicative of the structure of the standard BBS test where ‘easier’ tasks are performed at the beginning of test. These, however are less informative and have a lower predictive quality (see Table III). In the optimal ordering, these would appear later in the order with the more informative tasks appearing first.

The new order of BBS tasks as expressed in E-BBS was studied. First consider the physiotherapist training set and for simplicity focus on the initial task subset with the single BBS task #9 which was determined as the optimal starting task and set the confidence threshold to 100.

Reference is now made to FIGS. 11 and 12. FIG. 11. shows occurrence matrices depicting the ordering of BBS tasks in E-BBS. columns indicate the order in the E-BBS according to some embodiments of the invention; and FIG. 12. shows occurrence matrices depicting the ordering of BBS tasks in E-BBS trained on the automatic BBS scoring data according to some embodiments of the invention.

The columns of the matrix indicate the order in the E-BBS. Each row indicates a standard BBS task enumerated 1-14. The value in each matrix entry (i,j) indicates the proportion of times BBS task i appeared in an E-BBS sequence in position j counted across all E-BBS sequences produced over the test set.

FIG. 11 displays four occurrence matrices trained and tested on the physiotherapist data. Matrices a) to d) show results for selector methods 1 to 4 respectively. It can be seen that the number of BBS tasks used in the E-BBS sequences decreases along the order. This is due to the fact that for most patients the number of tasks required to reach the confidence threshold is much lower than 14 and the E-BBS evaluation is terminated before all 14 tasks are performed. Selector methods 1 and 2, which are nonadaptive, produce a constant sequence of E-BBS which is a permutation of the standard BBS. Selector methods 3 and 4 are adaptive and thus produce a different E-BBS sequence for each test.

However, it can be seen that the first 2 tasks in the sequence are always the same—task #9 and #11 (followed by #8 with high probability) and then show variability in the following tasks, with selector method 3 showing a wider variability than method 4. More interestingly is the fact that the initial part of the E-BBS sequence is similar across all 4 selector methods (all 4 matrices show initial BBS tasks 9, 11, 8 and even 7 with high values).

This indicates that regardless of whether the adaptive or constant E-BBS is used, the same BBS tasks will be invoked initially, implying that these tasks are predictive of the final assessment of risk of fall.

FIG. 12 displays similar occurrence matrices trained on the automatic scoring of BBS patients. Here too we see similar characteristics, albeit noisier. The common initial tasks in the E-BBS sequence on this data are BBS tasks 1, 12, 13. The distinction between this sequence and that obtained for the physiotherapist data is due to the fact that the automatic system introduces errors in the BBS scoring itself.

Thus, the tasks appearing early in the E-BBS are those that are predictive of fall risk as well as reliable in terms of BBS scoring. The outcome of this analysis implies that the E-BBS order of BBS tasks can be set as constant for the first 3 tasks (namely, tasks 9, 11, 8) followed by either the constant sequence determined by selector methods 1 and 2, or performed adaptively per patient using selector methods 3 or 4. Considering that most patient testing terminate early due to reaching the desired confidence level, the E-BBS sequence beyond the first 3-6 tasks is rare.

TABLE IV
E-BBS order of tasks. Task numbers are the standard BBS task numbers. Shaded cells are task steps
that may be determined. Adaptively per patient instead of following the set the sequence.

| | | Task | Task | Task | Task | Task | Task | Task | Task | Task | Task | Task | Task | Task | Task |
|---|---|---|---|---|---|---|---|---|---|---|---|---|---|---|---|
| Data | Method# | 1 | 2 | 3 | 4 | 5 | 6 | 7 | 8 | 9 | 10 | 11 | 12 | 13 | 14 |
| Physiotherapist | 1 | 9 | 11 | 8 | 7 | 5 | 13 | 10 | 1 | 2 | 3 | 4 | 6 | 12 | 14 |
| | 2 | 9 | 11 | 8 | 7 | 5 | 12 | 10 | 2 | 3 | 1 | 6 | 13 | 4 | 14 |
| Automatic | 1 | 1 | 12 | 13 | 7 | 14 | 4 | 8 | 2 | 3 | 5 | 6 | 9 | 10 | 11 |
| | 2 | 1 | 12 | 13 | 6 | 11 | 8 | 2 | 3 | 10 | 4 | 7 | 9 | 5 | 14 |

The orderings of tasks were summarized in the E-BBS testing in Table IV. Sequences are shown for selector methods 1 and 2 and for the physiotherapist data and the automatic scoring data. As described above the first 3 tasks are common to all E-BBS options, diverging only later. Shaded cells indicate task steps that can be followed using the adaptive selector method 3 or 4 instead of following the set sequence.

Reference is now made to FIG. 13, which is a block diagram depicting a computing device, which may be included within an embodiment of a system for predicting the risk of fall of a user, according to some embodiments.

Computing device 10 may include a processor or controller 2 that may be, for example, a central processing unit (CPU) processor, a chip or any suitable computing or computational device, an operating system 3, a memory 4, executable code 5, a storage system 6, input devices 7 and output devices 8. Processor 2 (or one or more controllers or processors, possibly across multiple units or devices) may be configured to carry out methods described herein, and/or to execute or act as the various modules, units, etc. More than one computing device 10 may be included in, and one or more computing devices 10 may act as the components of, a system according to embodiments of the invention.

Operating system 3 may be or may include any code segment (e.g., one similar to executable code 5 described herein) designed and/or configured to perform tasks involving coordination, scheduling, arbitration, supervising, controlling or otherwise managing operation of computing device 10, for example, scheduling execution of software programs or tasks or enabling software programs or other modules or units to communicate. Operating system 3 may be a commercial operating system. It will be noted that an operating system 3 may be an optional component, e.g., in some embodiments, a system may include a computing device that does not require or include an operating system 3.

Memory 4 may be or may include, for example, a Random Access Memory (RAM), a read only memory (ROM), a Dynamic RAM (DRAM), a Synchronous DRAM (SD-RAM), a double data rate (DDR) memory chip, a Flash memory, a volatile memory, a non-volatile memory, a cache memory, a buffer, a short term memory unit, a long term memory unit, or other suitable memory units or storage units. Memory 4 may be or may include a plurality of possibly different memory units. Memory 4 may be a computer or processor non-transitory readable medium, or a computer non-transitory storage medium, e.g., a RAM. In one embodiment, a non-transitory storage medium such as memory 4, a hard disk drive, another storage device, etc. may store instructions or code which when executed by a processor may cause the processor to carry out methods as described herein.

Executable code 5 may be any executable code, e.g., an application, a program, a process, task or script. Executable code 5 may be executed by processor or controller 2 possibly under control of operating system 3. Although, for the sake of clarity, a single item of executable code 5 is shown in FIG. 13, a system according to some embodiments of the invention may include a plurality of executable code segments similar to executable code 5 that may be loaded into memory 4 and cause processor 2 to carry out methods described herein.

Storage system 6 may be or may include, for example, a flash memory as known in the art, a memory that is internal to, or embedded in, a micro controller or chip as known in the art, a hard disk drive, a CD-Recordable (CD-R) drive, a Blu-ray disk (BD), a universal serial bus (USB) device or other suitable removable and/or fixed storage unit. Data related to AOI may be stored in storage system 6 and may be loaded from storage system 6 into memory 4 where it may be processed by processor or controller 2. In some embodiments, some of the components shown in FIG. 13 may be omitted. For example, memory 4 may be a non-volatile memory having the storage capacity of storage system 6. Accordingly, although shown as a separate component, storage system 6 may be embedded or included in memory 4.

Input devices 7 may be or may include any suitable input devices, components or systems, e.g., a detachable keyboard or keypad, a mouse and the like. Output devices 8 may include one or more (possibly detachable) displays or monitors, speakers and/or any other suitable output devices. Any applicable input/output (I/O) devices may be connected to Computing device 10 as shown by blocks 7 and 8. For example, a wired or wireless network interface card (NIC), a universal serial bus (USB) device or external hard drive may be included in input devices 7 and/or output devices 8. It will be recognized that any suitable number of input devices 7 and output device 8 may be operatively connected to Computing device 1 as shown by blocks 7 and 8.

A system according to some embodiments of the invention may include components such as, but not limited to, a plurality of central processing units (CPU) or any other suitable multi-purpose or specific processors or controllers (e.g., similar to element 2), a plurality of input units, a plurality of output units, a plurality of memory units, and a plurality of storage units.

A neural network, e.g. a neural network implementing machine learning (e.g., implementing ML model 15), may refer to an information processing paradigm that may include nodes, referred to as neurons, organized into layers, with links between the neurons. The links may transfer signals between neurons and may be associated with weights. A NN may be configured or trained for a specific task, e.g., pattern recognition or classification. Training a NN for the specific task may involve adjusting these weights based on examples. Each neuron of an intermediate or last layer may receive an input signal, e.g., a weighted sum of output signals from other neurons, and may process the input signal using a linear or nonlinear function (e.g., an activation function). The results of the input and intermediate layers may be transferred to other neurons and the results of the output layer may be provided as the output of the NN. Typically, the neurons and links within a NN are represented by mathematical constructs, such as activation functions and matrices of data elements and weights. A processor, e.g., CPUs or graphics processing units (GPUs), or a dedicated hardware device may perform the relevant calculations.

Therefore, provided herein are system and method which alleviate the workload in fall risk assessment. In one embodiment, provided herein are system and method applicable to any time-consuming battery of tests. In one embodiment, provided herein are system and method which comprise a computational mechanism, which automates the BBS fall assessment process and allows easy, non-invasive, and accessible assessment of fall risk. In one embodiment, provided herein are new system and method comprising multi depth-camera human motion tracking system.

Unless explicitly stated, the method embodiments described herein are not constrained to a particular order or sequence. Furthermore, all formulas described herein are intended as examples only and other or different formulas may be used. Additionally, some of the described method embodiments or elements thereof may occur or be performed at the same point in time.

While certain features of the invention have been illustrated and described herein, many modifications, substitutions, changes, and equivalents may occur to those skilled in the art. It is, therefore, to be understood that the appended claims are intended to cover all such modifications and changes as fall within the true spirit of the invention.

Various embodiments have been presented. Each of these embodiments may of course include features from other embodiments presented, and embodiments not specifically described may include various features described herein.

1. A computer-implemented method for predicting risk of fall of a user, comprising:
receiving temporal data related to a skeleton of the user, from at least two sensors;
reconstructing from the temporal data at least one of, a temporal 3D scene reconstruction, and a temporal three-dimensional (3D) skeleton reconstruction;
extracting spatio-temporal features from the 3D scene reconstruction or the 3D skeleton reconstruction;
introducing at least one spatio-temporal feature to a machine-learning (ML) model, wherein said ML model is trained to predict fall probability of a user based on said spatio-temporal feature; and
predicting fall probability of the user based on an output of the ML model.

2. The computer-implemented method of claim 1, wherein training the ML model comprises:
receiving a training dataset, comprising a plurality of spatio-temporal features, wherein the plurality of spatio-temporal features was received by:
receiving a three-dimensional (3D) temporal data related to skeletons of a plurality of users, from the at least two sensors;
reconstructing from the temporal data at least one of: a plurality of temporal 3D scene reconstructions, and a plurality of 3D skeleton reconstructions;
extracting the plurality spatio-temporal features from the plurality of 3D scene reconstructions or the plurality of 3D skeleton reconstructions;
receiving a set of fall-related labels, corresponding to the plurality of spatio-temporal features; and
training the ML model based on the training dataset, to predict fall probabilities, using the set of fall-related labels as supervisory data.

3. The computer-implemented method of claim 2, wherein each fall-related label includes a fall probability received from a professional via a user device.

4. The computer-implemented method of claim 3, wherein each fall-related label is related to one or more spatio-temporal features received when a user was conducting at least one Berg Balance Scale (BBS) task.

5. The computer-implemented method according to claim 1, wherein the at least two sensors are selected from two video cameras located at different angles with respect to the user, one video camera and at least one positioning sensor located on a body of the user and an array of positioning sensors located on the body of the user.

6. The computer-implemented method according to claim 1, wherein the 3D temporal data related to a skeleton of the user is received while the user is performing at least one BBS task.

7. The computer-implemented method of claim 6, further comprising: determining a number and type of additional BBS tasks the user is required to perform based on a fall probability given to the at least one BBS task.

8. The computer-implemented method of claim 7, wherein the number and the type of the BBS tasks are determined such that an accuracy of the prediction is at least 85% with respect to a training dataset.

9. The computer-implemented method of claim 7, further comprising:
determining the order of performing the BBS tasks based on previously performed BBS tasks.

10. The computer-implemented method according to claim 6, wherein the at least one fall probability is predicated from 3D temporal data received while the user is performing up to 6 BBS tasks.

11. (canceled)

12. A system for predicting risk of fall of a user, comprising:
at least two sensors; and
at least one computing device configured to:
receive a temporal data related to a skeleton of the user, from the at least two sensors;
reconstruct from the temporal data at least one of, a temporal 3D scene reconstruction and a temporal three-dimensional (3D) skeleton reconstruction;
extract spatio-temporal features from the 3D scene reconstruction or the 3D skeleton reconstruction;
introduce at least one spatio-temporal feature to a machine-learning (ML) model, wherein said ML model is trained to predict fall probability of a user based on said spatio-temporal feature; and
predict at least one fall probability of the user based on an output of the ML model.

13. The system of claim 12, wherein the at least one computing device is configured to train the ML model to:
receive a training dataset, comprising a plurality of spatio-temporal features, wherein the plurality of spatio-temporal features was received, by:
receiving a three-dimensional (3D) temporal data related to skeletons of a plurality of users, from the at least two sensors;
reconstructing from the temporal data at least one of, a plurality of temporal 3D scene reconstructions or a plurality of 3D skeleton reconstructions;
extracting the plurality spatio-temporal features from the plurality of 3D scene reconstructions or the plurality of 3D skeleton reconstructions;
receive a set of fall-related labels, corresponding to the plurality of spatio-temporal features; and
train the ML model based on the training dataset, to predict fall probabilitys, using the set of fall-related labels as supervisory data.

14. The system of claim 13, wherein each fall-related label includes a fall probability received from a professional via a user device.

15. The system of claim 14, wherein each fall-related label is related to one or more spatio-temporal features received when a user was conducting at least one Berg Balance Scale (BBS) task.

16. The system according to claim 12, wherein the at least two sensors are selected from: two video cameras located at different angles with respect to the user, one video camera and at least one positioning sensor located on a body of the user and an array of positioning sensors located on the body of the user.

17. The system according to claim 12, wherein the 3D temporal data related to a skeleton of the user is received while the user is performing at least one BBS task.

18. The system of claim 17, wherein the at least one computing device is configured to:
determine a number and type of BBS tasks the user is required to perform based on a fall probability given to the at least one BBS task.

19. The system of claim 18, wherein the number and the type of the BBS tasks are determined such that an accuracy of the prediction is at least 85% with respect to the training dataset.

20. The system of claim 18, wherein the at least one computing device is configured to:
determine the order of performing the BBS tasks based on previously preformed BBS tasks.

21. The system according to claim 17, wherein the at least one fall probability is predicated from 3D temporal data received while the user is performing up to 6 BBS tasks.

22. (canceled)
